(19) United States
(12) Patent Application Publication
(10) Pub. No.: US 2018/0132841 A1
(43) Pub. Date: May 17, 2018

(54) SOFT ANCHOR WITH TUBULAR SHEATH HAVING FLARED ENDS

(71) Applicant: Arthrex, Inc., Naples, FL (US)

(72) Inventor: Peter J. Dreyfuss, Naples, FL (US)

(21) Appl. No.: 15/349,067

(22) Filed: Nov. 11, 2016

(51) Int. Cl. A61B 17/04

(52) U.S. Cl. CPC A61B 17/0401; A61B 2017/0406; A61B 2017/042; A61B 2017/0414; A61B 2017/0409

(57) ABSTRACT

A soft anchor for tissue repair that includes a flexible sheath that has first and second flared open ends and a tubular middle portion therebetween. A method of tissue repair includes loading the soft anchor with a flexible strand; installing the pre-loaded soft anchor into a pre-drilled hole in bone; and tensioning at least one free end of the flexible strand thereby rolling the first and second flared open ends and bunching the flexible sheath to fix the soft anchor in the hole in bone.

### FIELD OF THE INVENTION

The present invention relates to a soft anchor used for attachment of tissue to bone, and more particularly to a soft anchor that has flared ends for improved fixation in bone.

### BACKGROUND OF THE INVENTION

Various types of anchors have been developed for securing soft tissue to bone. For example, commonly assigned U.S. Pat. No. 9,463,011 to Dreyfuss et al, the subject matter of which is herein incorporated by reference, discloses a soft anchor for use in tissue repair. A need exists for a soft anchor with improved fixation in bone.

### SUMMARY OF THE INVENTION

Accordingly, the present invention provides a soft anchor for tissue repair that includes a flexible sheath configured to receive a flexible strand. The flexible sheath has a first flared open end, a second flared open end opposite the first flared open end, and a tubular middle portion between the first and second flared open ends. Each of the first and second flared ends has a diameter that is larger than a diameter of the tubular middle portion. At least one flexible strand, such as a suture, is received in the flexible sheath of the soft anchor.

The present invention also provides a method of tissue repair comprising the steps of providing a soft anchor with a flexible sheath that has first and second flared open ends and a tubular middle portion therebetween; loading the soft anchor with at least one flexible strand by extending the flexible strand through the flexible sheath of the soft anchor; installing the pre-loaded soft anchor into a pre-drilled hole in bone; and tensioning at least one free end of the flexible strand thereby rolling the first and second flared open ends and bunching the flexible sheath to fix the soft anchor in the hole in bone.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing figures:

FIG. 1 is an elevational view of a soft anchor according to an exemplary embodiment of the present invention;

FIG. 2 is a schematic elevational view of the soft anchor illustrated in FIG. 1, showing the soft anchor being captured by an inserter for installing the soft anchor in a pre-drilled hole in bone, in accordance with a method of tissue repair of the present invention; and

FIG. 3 is a schematic elevational view of the soft anchor illustrated in FIG. 1, showing the soft anchor after installation in the pre-drilled bone hole, in accordance with a method of tissue repair of the present invention.

### DETAILED DESCRIPTION OF THE PREFERRED EMBODIMENTS

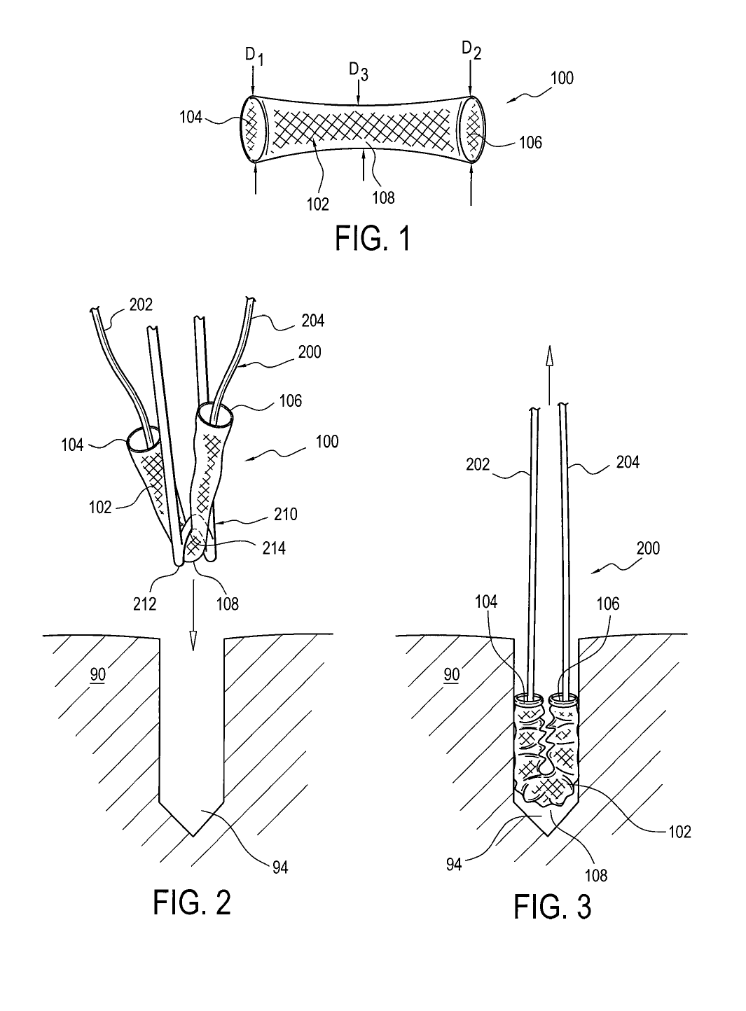

Referring to FIGS. 1-3, the present invention provides a soft anchor 100 for use in tissue repair procedures that is designed to ensure positive fixation in of the soft anchor 100 in bone.

Soft anchor 100 comprises a sheath 102 configured to receive at least one flexible strand 200, such as a suture. Sheath 102 is formed of flexible material, such as polyester or the like. Sheath 102 is preferably a suture braid or suture weave. Sheath may also be a knitted structure and/or formed of yarns, fibers, filaments, sutures or similar materials, or combinations of these materials. In a preferred embodiment, no portion of sheath 102 is rigid or semi-rigid or includes any rigid or semi-rigid structure, such as a flange or ring.

Sheath 102 has opposing open ends 104 and 106 and a middle portion 108 therebetween. Free ends 202 and 204 of flexible strand 200 preferably extend through open ends 104 and 106, respectively; however free ends 202 and 204 may extend through the length of sheath 102 at or near the sheath's open ends 104 and 106.

In accordance with a preferred embodiment, open ends 104 and 106 of sheath 102 have a flared shape, particularly with respect to middle portion 108 of sheath 102, which has a generally tubular shape. The diameters of the flared open ends 104 and 106 of sheath 102 are therefore larger than the diameter of middle portion 108. That is, a diameter D₁ of the sheath's flared open end 104 and a diameter D₂ of the sheath's other flared open end 106 are both larger than a diameter D₃ of the sheath's middle portion 108, as best seen in FIG. 1. The diameters D₁ and D₂ of the opposing open ends 104 and 106 may be substantially the same or different as long as both are larger than the diameter D₃ of middle portion 108. More specifically, diameters D₁ and D₂ are in the range of one and a half to three times as large as diameter D₃, preferably about twice as large as diameter D₃.

The method of tissue repair using the soft anchor of the present invention include the steps of pre-loading soft anchor 100 with flexible strand 200 by extending flexible strand 200 through flexible sheath 102 such that the strand's free ends 202 and 204 extend through the sheath's flared open ends 104 and 106, respectively. Once soft anchor 100 is pre-loaded with flexible strand 200 to form an anchor assembly, the pre-loaded soft anchor may be installed in a pre-drilled hole/socket 94 in the bone 90. To do so, an instrument, such as an inserter 210, may be used to capture and insert soft anchor 100, as best seen in FIG. 2. In a preferred embodiment, inserter 210 includes a forked tip 212 having a notch 214 sized to capture and receive the middle portion 108 of soft anchor 100. The pre-loaded soft anchor may then be pushed into the bone hole 94 using inserter 210 such that middle portion 108 of soft anchor 100 enters the bone hole 94 first with the flared open ends 104 and 106 being folded back and following the middle portion 108. A mallet may be used on the end of insert 210 opposite forked tip 212 to assist in pushing the pre-loaded soft anchor into the bone hole 94.

Once the pre-loaded soft anchor is inserted in the bone hole 94, inserter 210 is removed and soft anchor 100 takes on a generally U-shape and the free ends 202 and 204 of flexible strand 200 extend outside of the bone hole 94, as best seen in FIG. 3. Strand free ends 202 and 204 may extend through or around soft tissue to approximate the same to bone 90 in any known manner, either before or after the soft anchor 100 is installed in the bone hole 94. By tensioning one or both of the free ends 202 and 204 of flexible strand 200 when securing the tissue to bone 90, soft anchor 100 will roll and bunch up, thereby occupying more room in the bone hole 94 for improved fixation with the walls 90 of the bone hole 94. Flared open ends 104 and 106 of sheath 102 in particular will roll back toward middle portion 108 when tensioning the strand's free ends 202 and 204 due to the wider diameters of flared open ends 104 and 106. Also, the material of sheath 102, such as a braid or weave, may assist with anchoring of soft anchor 100 in the bone hole 94 because of its loose composition together with the trabecular cancellous nature of the bone creates a fixation therebetween.

While a particular embodiment has been chosen to illustrate the invention, it will be understood by those skilled in the art that various changes and modifications can be made therein without departing from the scope of the invention as defined in the appended claims.

What is claimed is:

1. A soft anchor for tissue repair, comprising:
a flexible sheath configured to receive a flexible strand, the flexible sheath having a first flared open end, a second flared open end opposite the first flared open end, and a tubular middle portion between the first and second flared open ends,
wherein each of the first and second flared ends has a diameter that is larger than a diameter of the tubular middle portion.

2. A soft anchor of claim 1, wherein the flexible sheath is formed of a suture braid or suture weave.

3. A soft anchor of claim 1, wherein the diameter of the first flared end and the diameter of the second flared end are substantially the same.

4. A soft anchor of claim 1, wherein the diameters of the first and second flared ends are at least twice the diameter of the tubular middle portion.

5. A soft anchor of claim 1, wherein no portion of the flexible sheath is rigid or semi-rigid.

6. An anchor assembly for tissue repair, comprising:
a soft anchor comprising a flexible sheath, the flexible sheath having a first flared open end, a second flared open end opposite the first flared open end, and a tubular middle portion between the first and second flared open ends, each of the first and second flared ends having a diameter that is larger than a diameter of the tubular middle portion; and
at least one flexible strand received in the flexible sheath of the soft anchor.

7. An anchor assembly according to claim 6, wherein opposite free ends of the flexible strand extend through the first and second flared open ends, respectively, of the flexible sheath.

8. An anchor assembly according to claim 7, wherein the flexible strand is a suture.

9. An anchor assembly according to claim 6, wherein the flexible sheath is a suture braid or suture weave.

10. A method of tissue repair, comprising the steps of:
providing a soft anchor with a flexible sheath that has first and second flared open ends and a tubular middle portion therebetween;
loading the soft anchor with at least one flexible strand by extending the flexible strand through the flexible sheath of the soft anchor;
installing the pre-loaded soft anchor into a pre-drilled hole in bone; and
tensioning at least one free end of the flexible strand thereby rolling the first and second flared open ends and bunching the flexible sheath to fix the soft anchor in the hole in bone.

11. A method according to claim 10, wherein the step of installing the pre-loaded soft anchor includes capturing the pre-loaded soft anchor with an inserter and using the inserter to push the pre-loaded soft anchor into the hole in bone.

12. A method according to claim 11, wherein the inserter includes a forked tip that captures the tubular middle portion of the flexible sheath of the soft anchor.

13. A method according to claim 10, wherein the flexible strand extends through the first and second flared open ends of the flexible sheath.

14. A method of claim 10, wherein the flexible sheath is formed of a suture braid or suture weave.

15. A method claim 10, wherein diameters of the first and second flared open ends, respectively, are larger than a diameter of the tubular middle portion.

16. A method claim 15, wherein the diameter of the first flared end and the diameter of the second flared end are substantially the same.

17. A method claim 15, wherein the diameters of the first and second flared ends are about twice the diameter of the tubular middle portion.
